(19) United States
(12) Patent Application Publication
(10) Pub. No.: US 2004/0049240 A1
(43) Pub. Date: Mar. 11, 2004

(54) Electrical and/or magnetic stimulation therapy for the treatment of prostatitis and prostatodynia

(76) Inventors: Martin Gerber, Maple Grove, MN (US); Kimberly Oleson, Shoreview, MN (US)

Correspondence Address: Thomas F. Woods, Medtronic, Inc., MS LC340, 710 Medtronic Parkway, Minneapolis, MN, 55432-5604, US

(21) Appl. No.: 10/236,578

(22) Filed: Sep. 6, 2002

(51) Int. Cl. A61N 1/18

(52) U.S. Cl. 607/40

(57) ABSTRACT

Described are combinations of implantable and external means for providing pain relief to patients suffering from prostatitis, prostatalgia or prostatodynia which employ electrical and/or magnetic stimulation to effect such relief. Implantable medical electrical leads or wireless modules comprising one or more electrodes deliver electrical stimulation to a selected site of a patient adjacent, around or in one of the sacral nerve or branches or portions thereof, the pudendal nerve or branches or portions thereof, the hypogastric nerve or branches or portions thereof, the prostatic plexus nerve or branches or portions thereof, the sacral splanchnic nerve or branches or portions thereof, or the pelvic splanchnic nerve or branches or portions thereof in an amount or degree sufficient to provide at least partial relief from pain intramuscular lead system. Optionally, an external or internal drug pump may be employed in conjunction with the electrical stimulation provided to the patient.

### FIELD OF THE INVENTION

This invention relates to fully implantable devices and methods for treating prostatitis, prostatalgia and prostatodynia utilizing electrical stimulation and, optionally, the delivery of drugs.

### BACKGROUND

The prostate is a glandular and fibromuscular organ in the male, which lies immediately below the bladder and surrounds the urethra. Prostatitis, the third leading disease of the prostate, is a common urologic condition that many clinicians find difficult to treat effectively.

It is now well recognized that prostatitis occurs in several distinct forms or syndromes. The old classifications of these were: (1) acute bacterial prostatitis; (2) chronic bacterial prostatitis; (3) chronic nonbacterial prostatitis; and (4) prostatodynia. Acute bacterial prostatitis is an abrupt illness with marked genito-urinary tract (urinary tract infection) and systemic symptoms. Chronic bacterial prostatitis is recurrent infection of the prostate by pathogenic bacteria, and can occur in the absence of urinary tract infection or systemic symptoms, but patients typically have a history of recurrent urinary tract infections. The infection and excessive inflammatory cells are localized to prostatic secretions. Evaluations in urologic clinics have demonstrated that 5% to 10% of cases are known to be bacterial prostatitis, which leaves more than 90% of cases to be non-bacterial prostatitis or prostatodynia.

In 1995, the NIH convened a workshop on prostatitis and a new classification system was agreed upon. The new system is based on pain and symptomology, and defines four categories of prostatitis. The first, Category I, is acute bacterial prostatitis and is unchanged from the old classification system. Category II is chronic bacterial prostatitis, where bacteria are found in prostatic secretions according to standard microbiological methodology, and is also unchanged from the old classification system. Category III is chronic prostatitis/chronic pelvic pain syndrome. The definition of this category is based on “the presence of genito-urinary pain in the absence of uropathogenic bacteria detected by standard microbiological methodology.” Chronic prostatitis/chronic pelvic pain syndrome is divided into 2 subtypes: inflammatory (Category IIIA) based on the presence of leukocytes in expressed prostatic secretions or post-prostatic massage urine, or noninflammatory (Category IIIB) that has 0 to <10 leukocytes in similar specimens. Category IV is asymptomatic inflammatory prostatitis characterized by a lack of prostate symptoms and accounts for an elevated prostate-specific antigen (PSA) seen in some patients with prostatitis.

The main symptom of chronic prostatitis (category III) is pain, followed by variable voiding (urgency/frequency) and erectile or sexual dysfunction. Patients have symptoms such as painful ejaculation or pain in the penis, testicles, or scrotum; low back, rectal or perineal pain; pain along the inner aspects of the thighs; irritative or obstructive urinary symptoms; and decreased libido or impotence. As a rule, chronic non-bacterial prostatitis patients do not have recurrent urinary tract infections.

Chronic prostatitis is a major male health issue. The average urologist in the U.S. sees 173 prostatitis patients per year, of which one-third are newly diagnosed. The prevalence of prostatitis in the general male population is estimated to be 5-8.8%, and it has been estimated that about 2 million office visits per year are related to prostatitis. Self-reported history of prostatitis is as prevalent as 16%. Patients with chronic prostatitis experience a negative impact on quality of life comparable to patients with unstable angina, recent myocardial infarction, or active Crohn's disease. The average age of the prostatitis population is estimated at 50 years, is the most common urologic diagnosis in men under 50 years old and the third most common in men over 50 years old. The most common classification of prostatitis is chronic prostatitis/chronic pelvic pain syndrome (category II), which may include as many as 90% of all patients who meet the criteria of the condition. Despite the widespread prevalence of prostatitis, the diagnosis of chronic prostatitis represents a particular challenge since its diagnosis is often based on exclusion.

Prostatitis remains poorly understood despite its prevalence because it encompasses multiple diverse disorders that cause symptoms related to the prostate gland. The etiology of acute and chronic bacterial prostatitis is clearly defined, and is a result of pathogenic bacteria that may cause systemic symptoms or urinary tract infections. On the other hand, chronic prostatitis/chronic pelvic pain syndrome does not have a clearly defined etiology, and there are many theories about the cause of this disease.

Non-bacterial prostatitis, despite its name, may in fact often have a bacterial component. Although bacteria do not grow in a culture, leukocytosis (more than 10-20 white blood cells per field) may be found in prostatic secretions. This is true for Category IIIA. An infectious etiology has been reported by some authors who describe, for example, an onset of symptoms associated with acute urethritis coincident with sexual activity.

Others have reported a chemical inflammatory reaction in the prostate, suggesting the role of urine reflux into the prostatic ducts as an underlying mechanism that increases concentrations of creatinine, urate and white blood cells. These authors also suggest that allopurinol treatment may result in elevated prostate-specific antigen levels in prostatic fluid and serum of patients with non-bacterial prostatitis via an effect of allopurinol on the cellular genome.

Perhaps the most encompassing theory of chronic non-bacterial prostatitis involves a multifactorial mechanism initiated by a stimulus such as infection or trauma. An interrelated cascade of events may follow, including physical, chemical, immunologic or neurogenic components, resulting in a local response of inflammation and/or neurogenic injury.

In the absence of consistent or clear etiologies for chronic prostatitis/chronic pelvic pain syndrome, improvement in quality of life and a reduction in symptoms are the usual goals of therapy. The most common treatment of chronic prostatitis includes pharmacologic treatments (antibiotics, anti-inflammatory agents, alpha blockers, anti-spasmodics, analgesics, allopurinol, and muscle relaxants). Alpha blockers have successfully treated symptoms, although adverse event rates have been high. Muscle relaxants have shown significant improvement in small studies for category IIIB patients with sphincter dyssynergia or muscle spasm. Anti-inflammatory agents, such as pentosan polysulfate, have proven successful for approximately 40% of patients with category IIIA prostatitis.

Phytotherapeutic agents have demonstrated improvements in small studies for pain and irritative voiding. Other treatments include physiotherapy (such as biofeedback and pelvic muscle exercises) and various modalities of invasive and minimally invasive procedures (e.g., transurethral microwave therapy, transurethral incision of the bladder neck, hydrodistensions, acupuncture, electroneuromodulation, balloon dilation, YAG laser therapy and heat therapy). Repetitive prostatic massage is a popular treatment method due to the failure of consistent standard medical therapy to treat the condition. Lifestyle changes, such as meditation, discontinuation of bike riding, sitz-baths, dietary changes, and chiropractic therapy, are often utilized.

As a result of unknown etiology, unsure diagnosis, and treatment options that are often myriad and ineffective, chronic prostatitis is a “diagnosis of exclusion” and has a poor record of treatment success. Accordingly, the present invention is intended to provide solutions to the foregoing problems through improved and more effective methods of treating pain and other symptoms associated with chronic prostatitis, prostatalgia and prostatodynia.

### SUMMARY OF THE INVENTION

The present invention has certain objects. That is, various embodiments of the present invention provide solutions to one or more problems existing in the prior art, including the problems of: (a) chronic prostatitis, prostatalgia and prostatodynia being untreated or largely untreated disease states in many patients; (b) prostatitis, prostatalgia and prostatodynia causing pain which is untreatable or inadequately treatable in many patients; (c) many patients suffering chronic prostatitis, prostatalgia and prostatodynia being treated with drugs that cause one or more side effects; (d) patients suffering from chronic prostatitis, prostatalgia and prostatodynia having few available treatement alternatives; (e) drugs administered to patients suffering from chronic prostatitis, prostatalgia and prostatodynia being relatively unspecific to the areas or regions of patients' bodies which they affect; and (f) drug treatment for chronic prostatitis, prostatalgia and prostatodynia not being adequately responsive to changes in pain levels experience by patients.

Various embodiments of the present invention have certain advantages, including one or more of: (a) providing a lower cost alternative to drugs; (b) having no or fewer side effects than drugs; (c) providing a therapy which is reversible; (d) being targeted to a very specific portion of the body; (e) being controllable or modulatable by a patient; (f) in some embodiments of the present invention, no injections of drugs being necessary or desirable; (g) in some embodiments of the present invention, no ingestion of drugs being necessary or desirable; (h) providing continuous, as opposed to episodic, relief from pain; and (i) providing a therapy capable of being adjusted or modulated as pain levels change and the disease state progresses or changes.

Various embodiments of the present invention have certain features, including one or more of: (a) providing a patient or health care giver with the ability to control or modulate the amplitude, directionality, frequency and/or pulse width of the electrical and/or magnetic stimulation being provided by fully implantable means to one or more nerve sites within the patient to thereby reduce or eliminate the pain being experienced by the patient resulting from chronic prostatitis, prostatalgia or prostatodynia; (b) using fully implantable means, simultaneously or sequentially electrically and/or magnetically stimulating one, two or more appropriate nerves in a patient to reduce or eliminate the pain being experienced by the patient resulting from chronic prostatitis, prostatalgia or prostatodynia; (c) implanting an appropriate implantable pulse generator and one or more implantable medical electrical and/or electromagnetically/inductively-coupled leads near or at appropriate nerve stimulation sites in a patient, and subsequently electrically stimulating such sites in an amount or to a degree sufficient to provide relief from pain suffered as a result of chronic prostatitis, prostatalgia and prostatodynia; (c) in conjunction with using fully implantable means to simultaneously or sequentially electrically or magnetically stimulate one, two or more appropriate nerves in a patient, delivering a drug to a specific site within the patient's body using an implantable drug pump and associated catheter, to reduce or eliminate the pain being experienced by the patient resulting from chronic prostatitis, prostatalgia or prostatodynia.

### BRIEF DESCRIPTION OF THE DRAWINGS

These and other objects, features and advantages of the present invention will be more readily understood from the following detailed description of the preferred embodiments thereof, when considered in conjunction with the drawings, in which like reference numerals indicate identical structures throughout the several views, and wherein:

FIG. 1 is a simplified schematic view of one embodiment of INS (implantable nerve stimulator) 10 of the present invention having a lead positioned near a desired or target nerve or nerve portion 8.

FIG. 2 shows a block diagram illustrating some of the constituent components of INS 10 in accordance with one embodiment of the present invention.

FIG. 3 shows one embodiment of the present invention, where INS 10 is implanted in an upper buttock position in a patient and lead 16 is implanted near or adjacent to pudendal nerve 26 to thereby effect relief from pain attendant to prostatitis, prostatalgia or prostatodynia.

FIG. 4 shows another embodiment of the present invention, where INS 10 is implanted in an appropriate location within the patient and lead 16 is implanted near or adjacent to one or more of pelvic plexus nerve 28, vesical plexus nerve 30, prostatic plexus 32 and/or prostate gland 34 to thereby effect relief from pain attendant to prostatitis, prostatalgia or prostatodynia.

FIG. 5 shows a further embodiment of the present invention, where INS 10 is implanted in an appropriate location within the patient and lead 16 is implanted near or adjacent to one or more of prostatic plexus 32, prostate gland 34, hypogastric nerve 33, sacral nerves S1, S2, S3 and S4, pelvic splanchnic nerve 36 and/or pudendal nerve 26 to thereby effect relief from pain attendant to prostatitis, prostatalgia or prostatodynia.

FIGS. 6A through 6E show various embodiments of the distal end of lead 16 of the present invention.

FIG. 7 shows a flow diagram of one method of stimulating a nerve in accordance with the present invention for effecting pain relief from prostatitis, prostatalgia or prostatodynia in a patient.

### DESCRIPTIONS OF THE PREFERRED EMBODIMENTS

In the following descriptions of the preferred embodiments, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense. Instead, the scope of the present invention is to be defined in accordance with the appended claims.

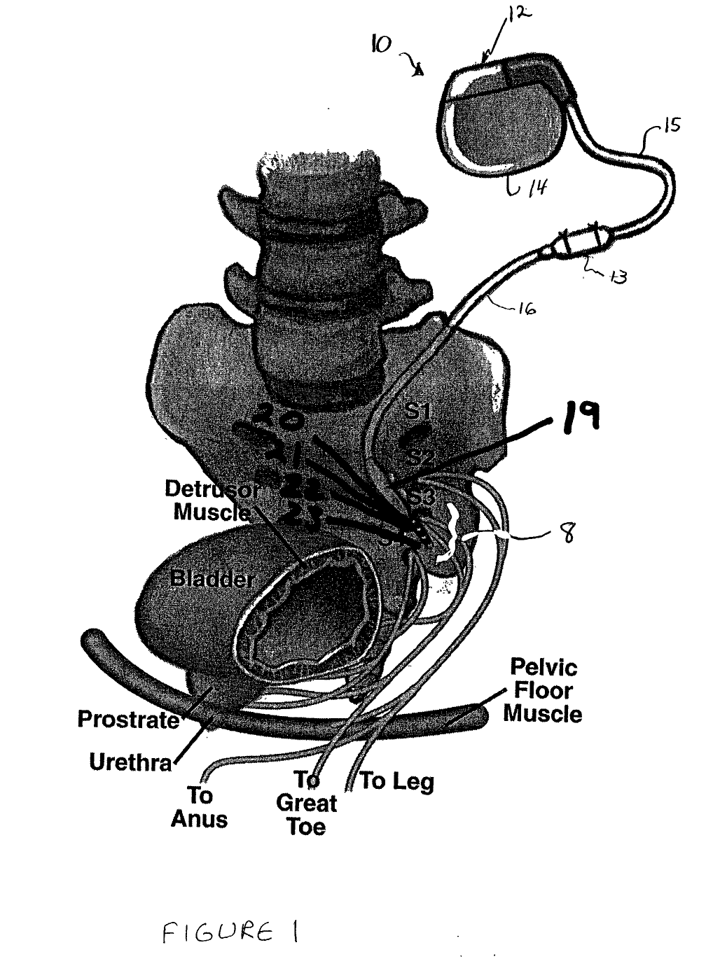

FIG. 1 is a simplified schematic view of one embodiment of INS (implantable nerve stimulator) 10 of the present invention having a lead positioned near a desired or target nerve or nerve portion 8. INS 10 shown in FIG. 1 is a implantable electrical stimulator comprising at least one implantable medical electrical lead 16 attached to hermetically sealed enclosure 14, lead 16 being implanted near desired or target nerve or nerve portion 8. Enclosure 14 is formed of a biocompatible material such as an appropriate metal alloy containing titanium. It is important to note that at least one more lead 18 (not shown in the drawings) may be employed in accordance with certain embodiments of the present invention, where multiple nerve target sites or portions are to be stimulated simultaneously or sequentially and/or where such multiple target sites or portions are incapable of being stimulated, or are difficult to stimulate, using a single lead even if the single lead contains multiple stimulation electrodes or arrays of stimulation electrodes.

Lead 16 provides electrical stimulation pulses to the desired nerve target sites or portions and thereby causes paresthesia, or the masking or blocking pain signals originating in or carried by a desired or target nerve or nerve portion located in the vicinity of the electrode(s) thereof. Leads 16 and 18 may have unipolar electrodes disposed thereon (where enclosure 14 is employed as an indifferent electrode) or may have bipolar electrodes disposed thereon, where one or more electrodes disposed on a lead are employed as the indifferent electrode. In one embodiment of the present invention, Lead 16 extends from lead connector 13, which in turn forms an integral portion of lead extension 15 connected at its proximal end to connector header module 12.

Leads 16 and 18 are preferably less than about 5 mm in diameter, and most preferably less than about 1.5 mm in diameter. Polyurethane is a preferred material for forming the lead body of leads 16 and 18, although other materials such as silicone may be employed. Electrical conductors extending between the proximal and distal ends of leads 16 and 18 for supplying electrical current to the electrodes are preferably formed of coiled, braided or stranded wires comprising an MP35N platinum-iridium alloy. Electrodes 20, 21, 22 and 23 may be ring electrodes, coiled electrodes, electrodes formed from portions of wire, barbs, hooks, spherically-shaped members, helically-shaped members, or may assume any of a number of different structural configurations well known in the art. Inter-electrode distances on leads 16 and 18 are preferably about 3 mm, but other inter-electrode distances may be employed such as about 1 mm, about 2 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 25 mm, about 30 mm. Preferred surface areas of electrodes 20, 21, 22 and 23 range between about 1.0 sq. mm and about 100 sq. mm, between about 2.0 sq. mm and about 50 sq. mm, and about 4.0 sq. mm and about 25 sq. mm. Preferred lengths of electrodes 20, 21, 22 and 23 range between about 0.25 mm and about 10 mm, between about 0.50 mm and about 8 mm, and about 1.0 mm and about 6 mm. Table 1 below shows representative values of the electrode surface areas and lengths for a Medtronic Model No. 3080 lead. Electrodes 20, 21, 22 and 23 are preferably formed of platinum, although other metals and metal alloys may be employed such as stainless steel or gold. Table 1: Medtronic Model No. 3080 Lead Electrode Surface Areas and Lengths

TABLE 1
Medtronic Model No. 3080 Lead Electrode Surface Areas and Lengths
Lead Model 3080

| | | | Electrode | Electrode | Electrode area per |
|---|---|---|---|---|---|
| | | | length mm | Area mm² | Lead 4 electrodes |
| Electrode | 0.118 | 2.9972 | | | |
| | inch | mm | | | |
| Lead | 0.050 | 1.27 | 0.5 | 2.0 | 8.0 |
| Diameter | inch | mm | | | |

| | | |
|---|---|---|
| 1 | 4.0 | 16.0 |
| 2 | 8.0 | 31.9 |
| 3 | 12.0 | 47.9 |
| 4 | 16.0 | 63.8 |
| 5 | 19.9 | 79.8 |
| 6 | 23.9 | 95.8 |
| 7 | 27.9 | 111.7 |
| 8 | 31.9 | 127.7 |
| 9 | 35.9 | 143.6 |
| 10 | 39.9 | 159.6 |

The distal portion of lead 16 extends to a target site or position near a desired nerve or nerve portion 8, and is preferably held in such position by lead anchor 19. Note that lead anchor 19 may assume any of a number of different structural configurations such one or more suture sleeves, tines, barbs, hooks, a helical screw, tissue in-growth mechanisms, adhesive or glue.

One, two, three, four or more electrodes 20, 21, 22 and 23 may be disposed at the distal end of lead 16 and/or lead 18. Electrodes 20, 21, 22 and 23 are preferably arranged in an axial array, although other types of arrays may be employed such as inter-lead arrays of electrodes between the distal ends of leads 16 and 18 such that nerves or nerve portions 8 disposed between leads 16 and 18 may be stimulated. Electrode configurations, arrays and stimulation patterns and methods similar to those disclosed by Holsheimer in U.S. Pat. No. 6,421,566 entitled “Selective Dorsal Column Stimulation in SCS, Using Conditioning Pulses,” U.S. Pat. No. 5,643,330 entitled “Multichannel Apparatus for Epidural Spinal Cord Stimulation” and U.S. Pat. No. 5,501,703 entitled “Multichannel Apparatus for Epidural Spinal Cord Stimulator,” the respective entireties of which are hereby incorporated by reference herein, may also be adapted or modified for use in the present invention. Electrode configurations, arrays, leads, stimulation patterns and methods similar to those disclosed by Thompson in U.S. Pat. No. 5,800,465 entitled “System and Method for Multisite Steering of Cardiac Stimuli,” the entirety of which is hereby incorporated by reference herein, may also be adapted or modified for use in the present invention to permit the steering of electrical fields. Thus, although FIG. 1 shows four electrodes located at the distal end of lead 16 near sacral nerve S3, other lead locations and electrode configurations are possible and contemplated in the present invention.

Leads 16 and 18 preferably range between about 4 inches and about 20 inches in length, and more particularly may be about 6 inches, about 8 inches, about 10 inches, about 12 inches, about 14 inches, about 16 inches or about 18 inches in length, depending on the location of the site to be stimulated and the distance of INS 10 from such site. Other lead lengths such as less than about 4 inches and more than about 20 inches are also contemplated in the present invention.

Typically, leads 16 and 18 are tunneled subcutaneously between the location of INS 10 and the location or site of the nerve or nerve portion that is to be stimulated. INS 10 is typically implanted in a subcutaneous pocket formed beneath the patient's skin according to methods well known in the art. Further details concerning various methods of implanting INS 10 and leads 16 and 18 are disclosed in the Medtronic Interstim Therapy Reference Guide published in 1999, the entirety of which is hereby incorporated by reference herein. Other methods of implanting and locating leads 16 and 18 are also contemplated in the present invention.

Some representative examples of leads 16 and 18 include MEDTRONIC nerve stimulation lead model numbers 3080, 3086, 3092, 3487, 3966 and 4350 as described in the MEDTRONIC Instruction for Use Manuals thereof, all hereby incorporated by reference herein, each in its respective entirety. Some representative examples of INS 10 include MEDTRONIC implantable electrical stimulator model numbers 3023, 7424, 7425 and 7427 as described in the Instruction for Use Manuals thereof, all hereby incorporated by reference herein, each in its respective entirety. INS 10 may also be constructed or operate in accordance with at least some portions of the implantable stimulators disclosed in U.S. Pat. No. 5,199,428 to Obel et al., U.S. Pat. No. 5,207,218 to Carpentier et al. or U.S. Pat. No. 5,330,507 to Schwartz, all of which are hereby incorporated by reference herein, each in its respective entirety.

In other embodiments of the present invention, leads 16 and 18 may be configured to electromagnetically or inductively couple with electromagnetic fields generated or radiated by an implantable or external pulse generator, thereby eliminating the requirement for a physical connection between IMD 10 (or an external generator) and lead 16 or 18. Accordingly, magnetic stimulation methods and devices are contemplated for use in some embodiments of the present invention.

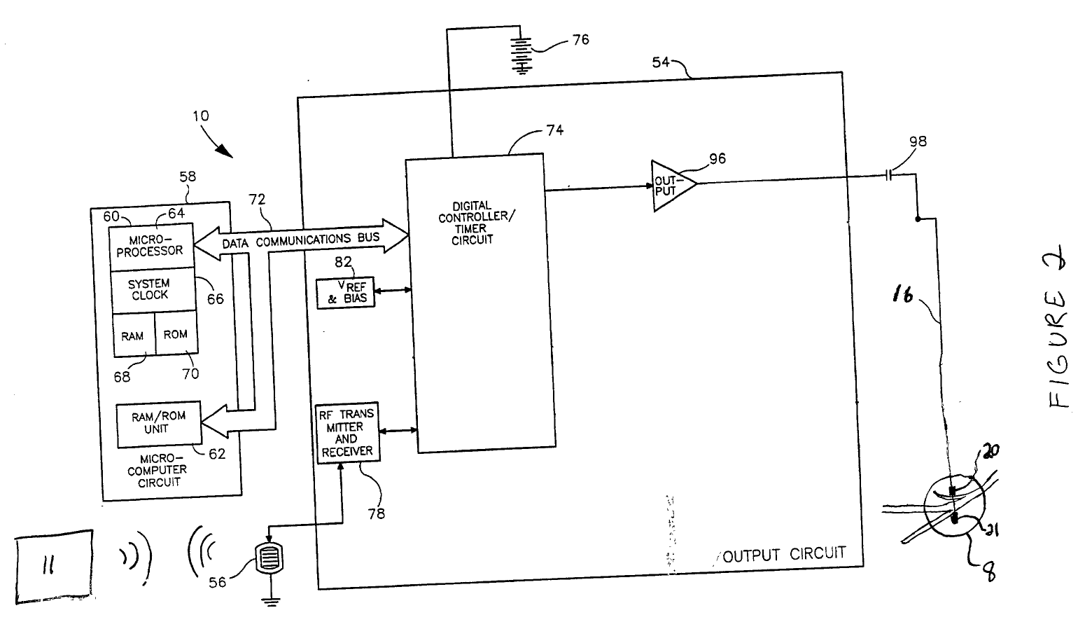

FIG. 2 shows a block diagram illustrating some of the constituent components of INS 10 in accordance with one embodiment of the present invention, where INS 10 is an implantable electrical stimulator having a microprocessor-based architecture. Other architectures of INS 10 are of course contemplated in the present invention, such as the logic or state machine architecture employed in the Medtronic Model Number 3023 INS. For the sake of convenience, INS 10 in FIG. 2 is shown with only one lead 16 connected thereto; similar circuitry and connections not shown in FIG. 2 apply generally to lead 18 and other additional leads not shown in the drawings. INS 10 in FIG. 2 is most preferably programmable by means of external programming unit 11. One such programmer is the commercially available Medtronic Model No. 7432 programmer, which is microprocessor-based and provides a series of encoded signals to INS 10, typically through a programming head which transmits or telemeters radio-frequency (RF) encoded signals to INS 10. Another suitable programmer is the commercially available Medtronic Model No. 8840 programmer, which is also microprocessor-based but features a touch control screen. Any of a number of suitable programming and telemetry methodologies known in the art may be employed so long as the desired information is transmitted to and from the implantable electrical stimulator 10.

As shown in FIG. 2, lead 16 is coupled to node 50 in INS 10 through input capacitor 52. Microcomputer circuit 58 preferably comprises on-board circuit 60 and off-board circuit 62. Circuit 58 may correspond to a microcomputer circuit disclosed in U.S. Pat. No. 5,312,453 to Shelton et al., hereby incorporated by reference herein in its entirety. On-board circuit 60 preferably includes microprocessor 64, system clock circuit 66 and on-board RAM 68 and ROM 70. Off-board circuit 62 preferably comprises a RAM/ROM unit. On-board circuit 60 and off-board circuit 62 are each coupled by data communication bus 72 to digital controller/timer circuit 74. Microcomputer circuit 58 may comprise a custom integrated circuit device augmented by standard RAM/ROM components.

Electrical components shown in FIG. 2 are powered by an appropriate implantable primary (i.e., non-rechargeable) battery power source 76 or secondary (i.e., rechargeable) battery power source 76. For the sake of clarity, the coupling of battery 76 to the various components of INS 10 is not shown in the Figures. Antenna 56 is connected to microcomputer circuit 58 via digital controller/timer circuit 74 and data communication bus 72 to permit uplink/downlink telemetry through RF transmitter and receiver telemetry unit 78. By way of example, telemetry unit 78 may correspond to that disclosed in U.S. Pat. No. 4,566,063 issued to Thompson et al. It is generally preferred that the particular programming and telemetry scheme selected permit the entry and storage of electrical stimulation parameters. The specific embodiments of antenna 56 and other telemetry circuitry presented herein are shown for illustrative purposes only, and are not intended to limit the scope of the present invention.

Continuing to refer to FIG. 2, V_{REF} and bias circuit 82 most preferably generate stable voltage reference and bias currents for analog circuits included in output circuit 54. Operating commands for controlling the timing of INS 10 are coupled by data bus 72 to digital controller/timer circuit 74, where digital timers and counters establish the specific stimulation parameters of INS 10 as well as various timing windows for controlling the operation of peripheral components disposed within input/output circuit 54.

Output pulse generator 96 provides pacing stimuli to the desired nerve or nerve portion through coupling capacitor 98 in response to a trigger signal provided by digital controller/timer circuit 74, when an externally transmitted stimulation command is received, or when a response to other stored commands is received. By way of example, output amplifier 96 may correspond generally to an output amplifier disclosed in U.S. Pat. No. 4,476,868 to Thompson, hereby incorporated by reference herein in its entirety. The specific embodiments of output amplifier 96 identified herein are presented for illustrative purposes only, and are not intended to be limiting in respect of the scope of the present invention. The specific embodiments of such circuits may not be critical to practicing some embodiments of the present invention so long as they provide means for generating an appropriate train of stimulating pulses to the desired nerve or nerve portion.

In various embodiments of the present invention, INS 10 may be programmably configured to operate so that it varies the rate at which it delivers stimulating pulses to the desired nerve or nerve portion 8 in response to one or more selected outputs being generated. INS 10 may further be programmably configured to operate so that it may vary the morphology of the stimulating pulses it delivers. Numerous implantable electrical stimulator features and functions not explicitly mentioned herein may be incorporated into INS 10 while remaining within the scope of the present invention. Various embodiments of the present invention may be practiced in conjunction with one, two, three or more leads, or in conjunction with one, two, three, four or more electrodes.

It is important to note that leadless embodiments of the present invention are also contemplated, where one or more stimulation and/or sensing electrode capsules or modules are implanted at or near a desired nerve stimulation site, and the capsules or modules deliver electrical stimuli directly to the site using a preprogrammed stimulation regime, and/or the capsules or modules sense electrical or other pertinent signals. Such capsules or modules are preferably powered by rechargeable batteries that may be recharged by an external battery charger using well-known inductive coil or antenna recharging means, and preferably contain electronic circuitry sufficient to permit telemetric communication with a programmer, to deliver electrical stimuli and/or sense electrical or other signals, and to store and execute instructions or data received from the programmer. Examples of methods and devices that may be adapted for use in the wireless devices and methods of the present invention include those described in U.S. Pat. No. 6,208,894 to Schulman et al. entitled System of implantable devices for monitoring and/or affecting body parameters; U.S. Pat. No. 5,876,425 to Schulman et al. entitled Power control loop for implantable tissue stimulator; U.S. Pat. No. 5,957,958 to Schulman et al. entitled Implantable electrode arrays; and U.S. patent application Ser. No. 09/030,106 filed Feb. 25, 1998 to Schulman et al. entitled “Battery-Powered Patient Implantable Device.”

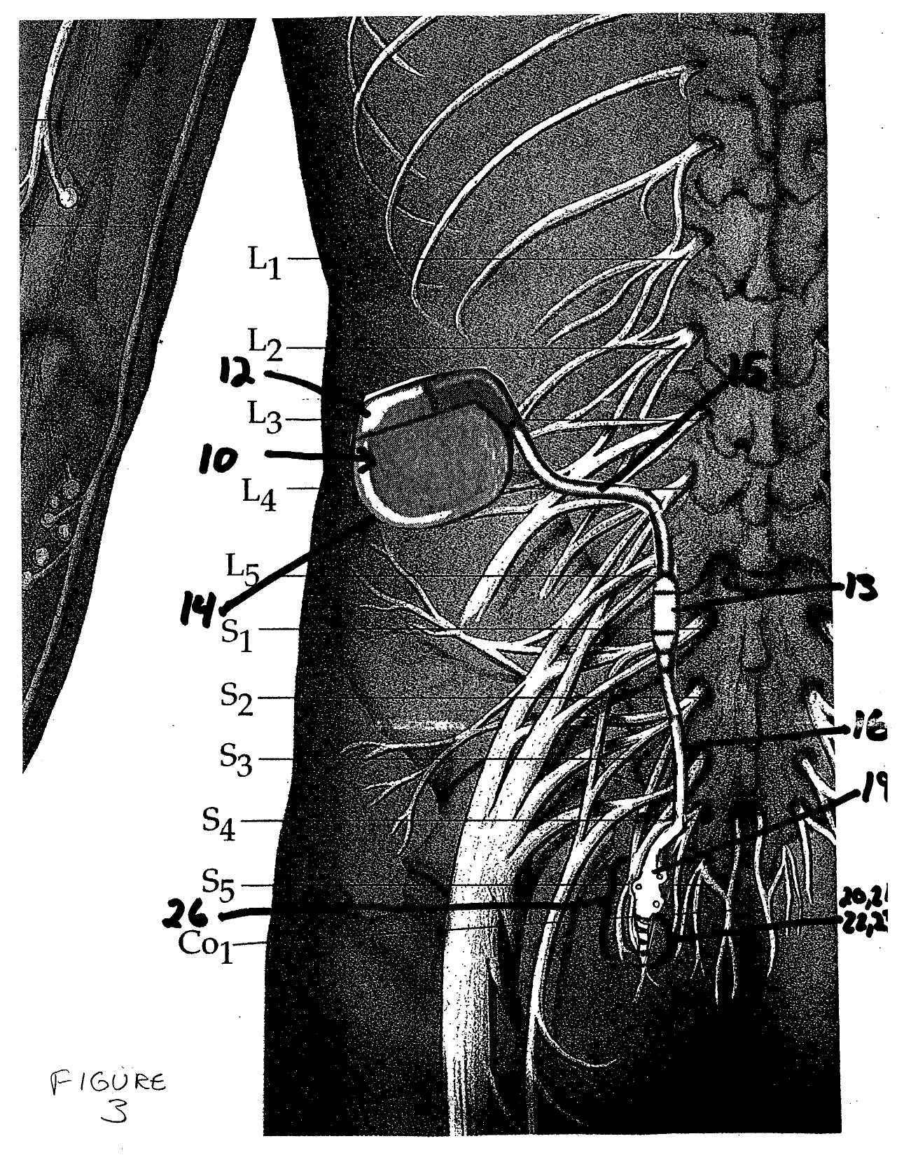

Referring now to FIG. 3, there is shown another embodiment of the system of the present invention, where INS 10 is implanted in an upper buttock position in a patient and lead 16 is implanted near or adjacent to pudendal nerve 26 to thereby effect relief from pain attendant to prostatitis, prostatalgia or prostatodynia. Such pain relief is effected as a result of electrical stimulation signals being delivered to or near pudendal nerve 26 by electrodes 20-23. One, two, three, four or more electrodes 20, 21, 22 and 23 may be disposed at the distal end of lead 16. FIG. 3 shows four electrodes located at the distal end of lead 16 near pudendal nerve 26. Other lead locations and electrode configurations are possible and contemplated in the present invention.

U.S. patent application Ser. No. 10/004,732 entitled “Implantable Medical Electrical Stimulation Lead Fixation Method and Apparatus” and Ser. No. 09/713,598 entitled “Minimally Invasive Apparatus for Implanting a Sacral Stimulation Lead” to Mamo et al., the respective entireties of which are hereby incorporated by reference herein, describe methods of percutaneously introducing leads 16 and 18 to a desired nerve stimulation site in a patient.

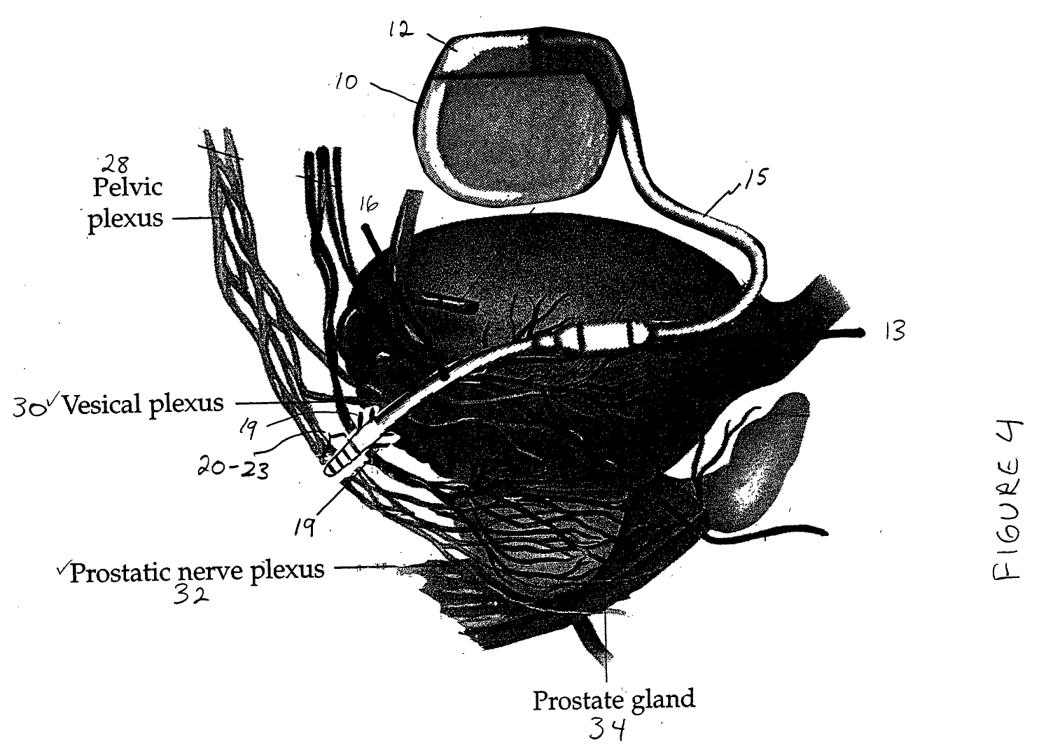

FIG. 4 shows another embodiment of the present invention, where INS 10 is implanted in an appropriate location within the patient and lead 16 is implanted near or adjacent to one or more of pelvic plexus nerve 28, vesical plexus nerve 30, prostatic plexus 32 and/or prostate gland 34 to thereby effect relief from pain attendant to prostatitis, prostatalgia or prostatodynia. Such pain relief is effected as a result of electrical stimulation signals being delivered to or near one or more of such nerves 28, 30, 32 and/or prostate gland 34 by electrodes 20-23. One, two, three, four or more electrodes 20, 21, 22 and 23 may be disposed at the distal end of lead 16. FIG. 4 shows four electrodes located at the distal end of lead 16 near pelvic plexus nerve 28. Lead locations and electrode configurations other than those explicitly shown and described here are of course possible and contemplated in the present invention. Lead anchors 19 are shown in FIG. 4 as a series of tines.

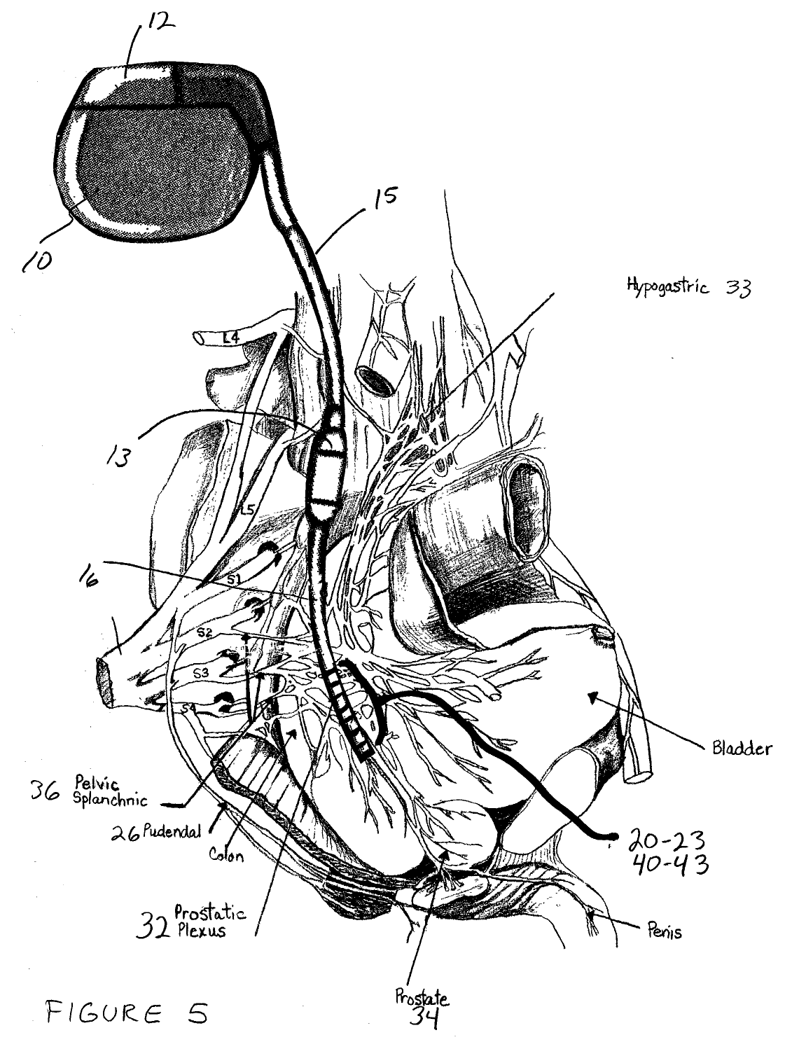

FIG. 5 shows a further embodiment of the present invention, where INS 10 is implanted in an appropriate location within the patient and lead 16 is implanted near or adjacent to one or more of prostatic plexus 32, prostate gland 34, hypogastric nerve 33, sacral nerves S1, S2, S3 and S4, pelvic splanchnic nerve 36 and/or pudendal nerve 26 to thereby effect relief from pain attendant to prostatitis, prostatalgia or prostatodynia. Such pain relief is effected as a result of electrical stimulation signals being delivered to or near to or near one or more of such nerves 32, 33, S1, S2, S3, S4, 36, 26 and/or prostate gland 34 by electrodes 20, 21, 22, 23, 40, 41, 42 and 43. One, two, three, four or more electrodes 20, 21, 22 and 23 may be disposed at the distal end of lead 16. FIG. 5 shows eight electrodes located at the distal end of lead 16 near prostatic plexus 32. Other lead locations and electrode configurations are possible and contemplated in the present invention.

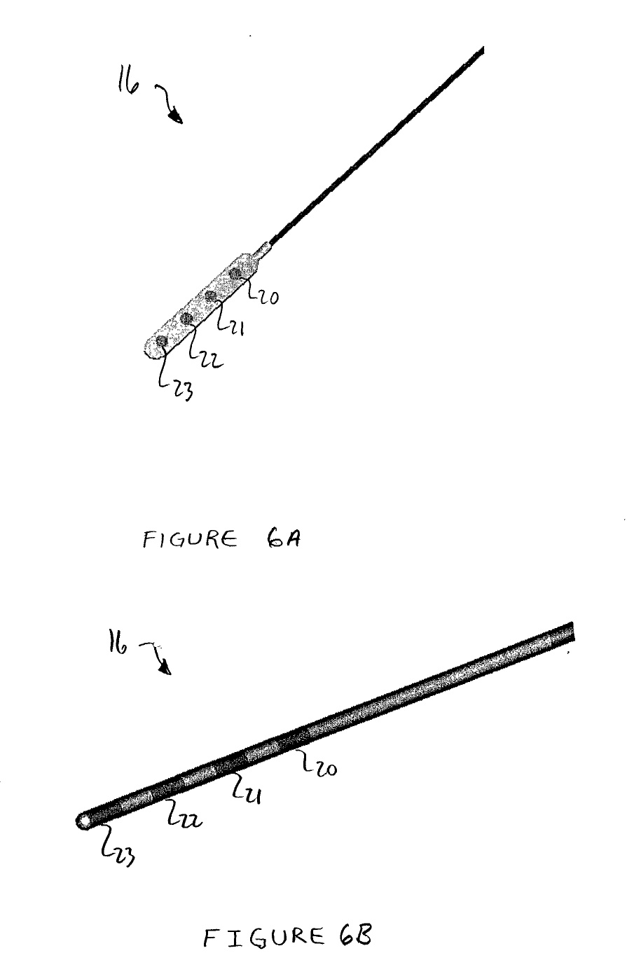

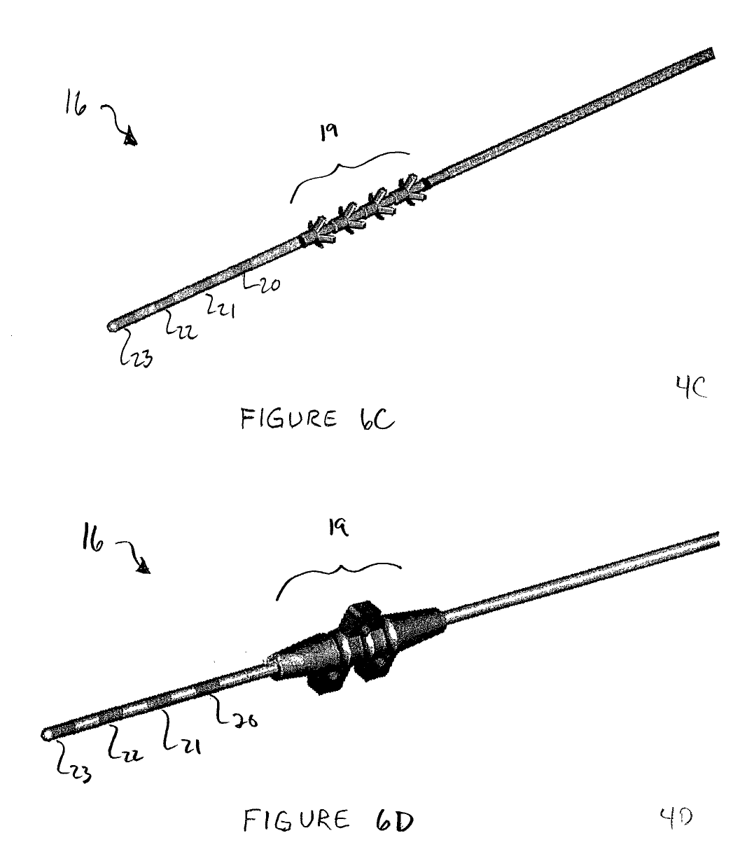

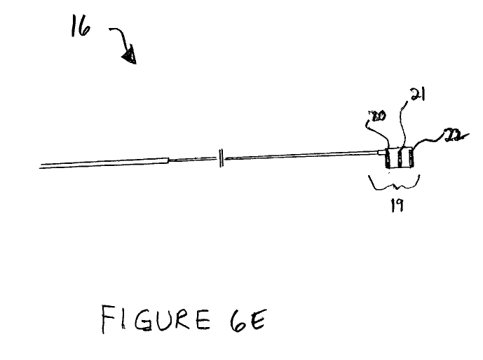

FIGS. 6A through 6D show various embodiments of the distal end of lead 16 of the present invention. In FIG. 6A, lead 16 is a paddle lead where electrodes 20-23 are arranged along an outwardly facing planar surface. Such a paddle lead is preferably employed to stimulate peripheral nerves. In FIG. 6B, lead 16 is a conventional quadrapolar lead having no pre-attached anchoring mechanism 198 where electrodes 20-23 are cylindrical in shape and extend around the circumference of the lead body. In FIG. 6C, lead 16 is a quadrapolar lead having tined lead anchors. The tines may be formed from flexible or rigid biocompatible materials in accordance with the application at hand. Representative examples of some tined and other types of leads suitable, adaptable or modifiable for use in conjunction with the systems, methods and devices of the present invention include those disclosed in U.S. patent application Ser. No. 10/004,732 entitled “Implantable Medical Electrical Stimulation Lead Fixation Method and Apparatus” and Ser. No. 09/713,598 entitled “Minimally Invasive Apparatus for Implanting a Sacral Stimulation Lead” to Mamo et al., and those disclosed in U.S. Pat. No. 3,902,501 to Citron entitled “Endocardial Lead,” U.S. Pat. No. 4,106,512 to Bisping entitled “Transvenously Implantable Lead,” U.S. Pat. No. 5,300,107 to Stokes entitled “Universal Tined Myocardial Pacing Lead.” In FIG. 6D, lead 16 is a quadrapolar lead having a pre-attached suture anchor. In FIG. 6E, lead 16 is a tri-polar cuff electrode, where cuff/anchor 19 is wrapped around desired nerve or nerve portion 8 to thereby secure the distal end of lead 16 to the nerve and position electrodes 20-22 against or near nerve or nerve portion 8. The Medtronic Model No. 3995 cuff electrode lead is one example of a lead that may be adapted for use in the present invention, the Instructions for Use manual of which is hereby incorporated by reference herein in its entirety.

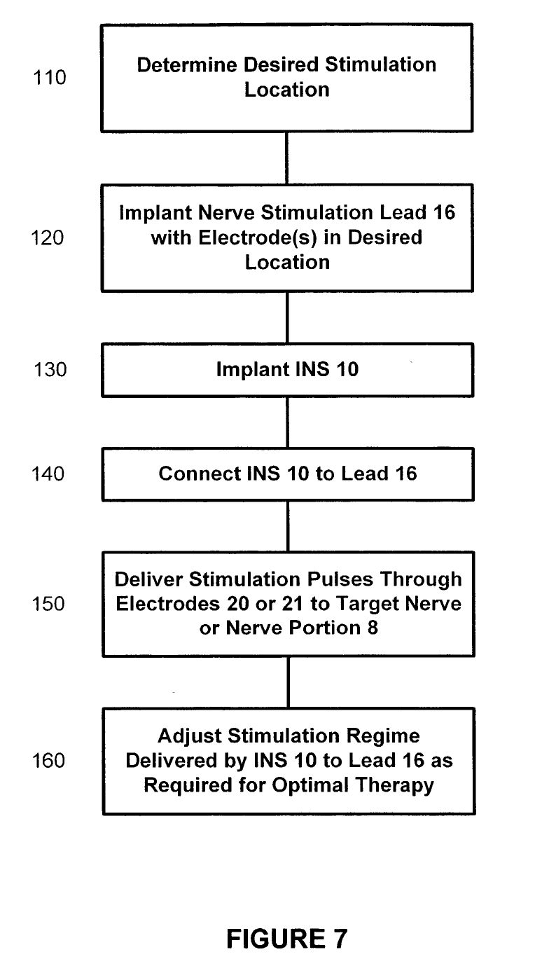

FIG. 7 shows a flow diagram for one method of stimulating a nerve portion, nerve or plurality of nerves in accordance one embodiment of the present invention, and thereby effecting the relief of pain resulting from prostatitis, prostatalgia or prostatodynia. In FIG. 7, step 110 is employed to determine one or more desired nerve stimulation locations near or at one or more of prostatic nerve plexus 32, hypogastric nerve 33, pelvic splanchnic nerve 36, pudendal nerve 26, prostate gland 34, vesicle nerve plexus 30, pelvic nerve plexus 28, sacral nerve S1, sacral nerve S2, sacral nerve S3, sacral nerve S4, and/or sacral nerve S5. Step 120 is employed to implant lead 16 and electrodes 20, 21, . . . n near or at the desired nerve stimulation site(s). (Note that as discussed above, methods of the present invention further contemplate the placement and implantation of multiple leads.) Step 130 is employed to implant INS 10 in an appropriate location within the patient such that the proximal end of lead 16 may be operably connected thereto and such that INS 10 is placed in such a location that discomfort and the risk of infection to the patient are minimized. Step 140 is employed to operably connect INS 10 to lead 16, which may or may not require the use of optional lead extension 15 and lead connector 13. In Step 150, INS 10 is activated and stimulation pulses are delivered to electrodes 20, 21, . . . n through lead 16 to the desired nerve stimulation location. In step 160, the electrical pulse stimulation parameters are adjusted to optimize the therapy delivered to the patient. Such adjustment may entail one or more of adjusting the number or configuration of electrodes or leads used to stimulate the selected location, pulse amplitude, pulse frequency, pulse width, pulse morphology (e.g., square wave, triangle wave, sinusoid, biphasic pulse, tri-phasic pulse, etc.), times of day or night when pulses are delivered, pulse cycling times, the positioning of the lead or leads, and/or the enablement or disablement of “soft start” or ramp functions respecting the stimulation regime to be provided.

Representative ranges of preferred electrical pulse stimulation parameters capable of being delivered by INS 10 through leads 16 and 18 include the following:

| | |
|---|---|
| Frequency: | Between about 50 Hz and about 100 Hz; |
| | Between about 10 Hz and about 250 Hz; and |
| | Between about 0.5 Hz and about 500 Hz. |
| Amplitude: | Between about 1 Volt and about 10 Volts; |
| | Between about 0.5 Volts and about 20 Volts; and |
| | Between about 0.1 Volts and about 50 Volts. |
| Pulse Width: | Between about 180 microseconds and about 450 |
| | microseconds; |
| | Between about 100 microseconds and about 1000 |
| | microseconds; and |
| | Between about 10 microseconds and about 5000 |
| | microseconds. |

In the event multiple signals are employed to stimulate a desired site, the spatial and temporal phase between the signals may be adjusted or varied to produce the desired stimulation pattern or sequence. That is, in the present invention beam forming and specific site targeting via electrode array adjustments are contemplated.

In addition, in the present invention it is contemplated that drugs be delivered to specific sites within a patient using well known fully implantable drug pump devices in combination with providing electrical stimulation to the nerves or nerve portions described above. According to such a method, the drug pump may be incorporated into the same housing as INS 10, or be separate therefrom in its own hermetically sealed housing. The drug catheter attached to the implantable drug pump through which the drug is delivered to the specific site may also be incorporated into lead 16 or 18, or may be separate therefrom. Drugs or therapeutic agents delivered in accordance with this method include, but are not limited to, antibiotics, pain relief agents such as demerol and morphine, radioactive or radiotherapeutic substances or agents for killing or neutralizing cancer cells, genetic growth factors for encouraging the growth of healthy tissues. Furthermore, localized delivery of anti-inflammatory agents, steroidal agents, antispasmodic agents, and paralyzing/denervation agents (such as botulism toxoid, etc) is contemplated in the present invention.

Hereby incorporated by reference herein in its entirety is U.S. Patent Application Number 20020082665A1 to Haller et al. published Jun. 27, 2002 and entitled “System and Method of Communicating between an Implantable Medical Device and a Remote Computer System or Health Care Provider.” In the present invention it is further contemplated that the methods and devices described hereinabove be extended to include the communication system of Haller et al. for at least one of monitoring the performance of INS 10 and/or an implantable drug pump implanted within the body of a patient, monitoring the health of the patient and remotely delivering an electrical stimulation and/or drug therapy to the patient through INS 10 and/or the optional implantable drug pump, INS 10 or the implantable drug pump being capable of bi-directional communication with a communication module located external to the patient's body, the system comprising: (a) INS 10 and optionally the implantable drug pump; (b) the communication module; (c) a mobile telephone or similar device operably connected to the communication module and capable of receiving information therefrom or relaying information thereto; (e) a remote computer system, and (f) a communication system capable of bi-directional communication.

All patents and patent applications referenced herein are incorporated by reference herein, each in its respective entirety.

Although various embodiments of the present invention have been described in some detail herein, it will be understood that variations and modifications of the present invention not explicitly disclosed herein are contemplated and fall within the scope of the present invention. Such modifications and variations may include the substitution of elements or components which perform substantially the same function in substantially the same way to achieve substantially the same effect.

We claim:

1. A method for treating at least one of prostatitis, prostatalgia and prostatodynia in a patient, comprising:
providing an hermetically sealed implantable electrical pulse generator configured to provide at least one electrical stimulation pulse regime effective to treat at least partially at least one of prostatitis, prostatalgia and prostatodynia in the patient;
providing at least a first implantable medical electrical lead configured for implantation adjacent, around or in at least one of a sacral nerve or branches or portions thereof, a pudendal nerve or branches or portions thereof, a hypogastric nerve or branches or portions thereof, a prostatic plexus nerve or branches or portions thereof, a sacral splanchnic nerve or branches or portions thereof, a pelvic splanchnic nerve or branches or portions thereof, the first lead comprising proximal and distal ends and at least one electrode;
implanting the first lead in tissue of the patient adjacent, around or in one of the sacral nerve or branches or portions thereof, the pudendal nerve or branches or portions thereof, the hypogastric nerve or branches or portions thereof, the prostatic plexus nerve or branches or portions thereof, the sacral splanchnic nerve or branches or portions thereof, or the pelvic splanchnic nerve or branches or portions thereof;
operably connecting the proximal end of the at least first lead to the implantable pulse generator;
implanting the implantable pulse generator within the patient; and
delivering electrical stimulation pulses from the implantable pulse generator to at least a portion of the tissue of the patient through the at least first lead and electrode, the pulses being provided in accordance with the electrical stimulation pulse regime and providing to the patient at least partial relief from pain resulting from at least one of prostatitis, prostatalgia and prostatodynia.

2. The method of claim 1, wherein the at least first lead is selected from the group consisting of a unipolar lead, a bipolar lead, a tri-polar lead, a quadrapolar lead, and a multi-polar lead.

3. The method of claim 1, wherein the at least first lead is selected from the group consisting of a beam steering lead comprising multiple electrodes and a lead comprising multiple electrodes disposed in an areal pattern on a planar or curved surface.

4. The method of claim 1, wherein the at least first lead is selected from the group consisting of a cuff lead, a paddle lead, a tined lead, a lead having an active fixation device or member disposed thereon, attached thereto or forming a portion thereof.

5. The method of claim 1, wherein the at least first lead is selected from the group consisting of a suture sleeve, a barb, a helical screw, a hook and a tissue in-growth mechanism.

6. The method of claim 1, wherein the at least first lead further comprises one or more electrodes configured to operate in conjunction with an electrically conductive portion of the implantable pulse generator acting as an indifferent electrode.

7. The method of claim 1, further comprising providing, implanting, operably connecting and delivering electrical stimuli from a second implantable medical electrical lead configured for implantation adjacent, around or in at least one of a sacral nerve or branches or portions thereof, a pudendal nerve or branches or portions thereof, a hypogastric nerve or branches or portions thereof and a prostatic plexus nerve or branches or portions thereof of the patient, wherein the second lead comprises proximal and distal ends and at least one electrode.

8. The method of claim 7, further comprising delivering the electrical pulses through tissue disposed between the electrodes located on the first and second leads.

9. The method of claim 1, wherein the electrical stimulation pulses that are delivered to the desired nerve target sites or portions cause paresthesia, or the masking or blocking pain signals originating in or carried by a desired or target nerve or nerve portion located in the vicinity of the at least one electrode.

10. The method of claim 1, further comprising providing a lead extension, operably connecting same between the proximal end of the at least first lead and the implantable pulse generator, and delivering the electrical stimulation pulses through the lead extension.

11. The method of claim 1, wherein the first lead is selected from the group consisting of a lead comprising a lead body less than about 5 mm in diameter, a lead comprising a lead body less than about 1.5 mm in diameter, a lead having a lead body comprising polyurethane or silicone, a lead comprising electrical conductors disposed within the body thereof and extending between the proximal and distal ends of the lead wherein the conductors are formed of coiled, braided or stranded wires, and a lead comprising at least one ring electrode, at least one coiled electrode, at least one button electrode, at least one electrode formed from a portion of wire, a barb or a hook, a spherically-shaped electrode, and a helically-shaped electrode.

12. The method of claim 1, wherein an inter-electrode distance of the first lead is selected from the group consisting of about 1 mm, about 2 mm, about 3 mm, about 4 mm, about 5 mm, about 6 mm, about 7 mm, about 8 mm, about 9 mm, about 10 mm, about 12 mm, about 14 mm, about 16 mm, about 18 mm, about 20 mm, about 25 mm, and about 30 mm.

13. The method of claim 1, wherein the at least one electrode of the first lead has an electrode surface area ranging between about 1.0 sq. mm and about 100 sq. mm, between about 2.0 sq. mm and about 50 sq. mm, or between about 4.0 sq. mm and about 25 sq. mm.

14. The method of claim 1, wherein the distance between the proximal and distal ends of the at least first lead is selected from the group consisting of less than about 4 inches, about 4 inches, about 6 inches, about 8 inches, about 10 inches, about 12 inches, about 14 inches, about 16 inches about 18 inches, about 20 inches and more than about 20 inches.

15. The method of claim 1, wherein the implantable pulse generator comprises an electronic circuitry architecture selected from the group consisting of a microprocessor-based architecture, a logic architecture and a state machine architecture.

16. The method of claim 1, further comprising providing an external programming unit and effecting telemetric communication between the programming unit and the implantable pulse generator.

17. The method of claim 1, wherein the implantable pulse generator further comprises at least one of a primary battery power source and a secondary battery power source.

18. The method of claim 1, wherein the implantable pulse generator is configurable so as to permit at least one of the frequency, rate, amplitude, phase, width and morphology of the pulses generated and delivered thereby to be varied programmably by a user.

19. The method of claim 1, wherein the at least first lead is configured for percutaneous introduction and implantation within the patient.

20. The method of claim 1, wherein the implantable pulse generator and the at least first lead are capable of generating and delivering electrical pulses having frequencies ranging between about 50 Hz and about 100 Hz, between about 10 Hz and about 250 Hz, and between about 0.5 Hz and about 500 Hz.

21. The method of claim 1, wherein the implantable pulse generator and the at least first lead are capable of generating and delivering electrical pulses having amplitudes ranging between about 1 Volt and about 10 Volts, between about 0.5 Volts and about 20 Volts, and between about 0.1 Volts and about 50 Volts.

22. The method of claim 1, wherein the implantable pulse generator and the at least first lead are capable of generating and delivering electrical pulses having pulse widths ranging between about 180 microseconds and about 450 microseconds, between about 100 microseconds and about 1000 microseconds, and between about 10 microseconds and about 5000 microseconds.

23. The method of claim 1, wherein the implantable pulse generator and the at least first lead and at least a second lead are capable of generating and delivering electrical pulses having varying spatial or temporal phases.

24. The method of claim 1, wherein the electrical stimulation pulse regime provided to the patient is effective in providing at least one of urinary urgency relief and urinary frequency relief.

25. The method of claim 1, wherein the electrical stimulation pulse regime provided to the patient is effective in providing relief from sexual dysfunction.

26. The method of claim 1, further comprising delivering a drug to the patient and delivering the electrical stimulation regime.

27. The method of claim 22, further comprising providing, implanting and activating an implantable drug pump for providing the drug to the patient.

28. The method of claim 1, further comprising providing at least one sensor to sense a physical condition, and adjusting the stimulation regime or parameters based on the sensed condition.

29. A method for treating at least one of prostatitis, prostatalgia and prostatodynia in a patient, comprising:
providing an electrical pulse controller configured to provide control or programming signals by wireless means to a fully implantable leadless module, the leadless module, in response to signals provided by the electrical pulse controller, providing least one electrical stimulation pulse regime effective to treat at least partially at least one of prostatitis, prostatalgia and prostatodynia in the patient, the leadless module further being configured for implantation adjacent, around or in at least one of a sacral nerve or branches or portions thereof, a pudendal nerve or branches or portions thereof, a hypogastric nerve or branches or portions thereof, a prostatic plexus nerve or branches or portions thereof, a sacral splanchnic nerve or branches or portions thereof, a pelvic splanchnic nerve or branches or portions thereof, the leadless module comprising at least one stimulation electrode;
implanting the leadless module in tissue of the patient adjacent, around or in one of the sacral nerve or branches or portions thereof, the pudendal nerve or branches or portions thereof, the hypogastric nerve or branches or portions thereof, the prostatic plexus nerve or branches or portions thereof, the sacral splanchnic nerve or branches or portions thereof, or the pelvic splanchnic nerve or branches or portions thereof;
effecting wireless communication between the leadless module and electrical pulse controller to at least one of program and control the electrical stimulation regime to be provided by the leadless module to the patient; and
delivering electrical stimulation pulses from the leadless module to at least a portion of the tissue of the patient through the at least one electrode, the pulses being provided in accordance with the electrical stimulation pulse regime programmed or controlled by the electrical pulse controller and providing to the patient pain relief from at least one of prostatitis, prostatalgia and prostatodynia.

30. The method of claim 29, further comprising providing a rechargeable battery in the leadless module for electrically powering same.

31. The method of claim 30, further comprising recharging the rechargeable battery using a battery charger external to the patient which inductively couples to the battery.

32. The method of claim 29, further comprising providing an external electrical pulse controller.

33. The method of claim 29, further comprising providing an implantable electrical pulse controller.

34. The method of claim 29, further comprising providing a plurality of leadless modules, each such module comprising at least one stimulation electrode.

35. The method of claim 34, further comprising implanting the plurality of leadless modules in tissue of the patient adjacent, around or in one of the sacral nerve or branches or portions thereof, the pudendal nerve or branches or portions thereof, the hypogastric nerve or branches or portions thereof, the prostatic plexus nerve or branches or portions thereof, the sacral splanchnic nerve or branches or portions thereof, or the pelvic splanchnic nerve or branches or portions thereof.

36. A method for treating pain in patients suffering from prostatitis, prostatalgia or prostatodynia, comprising:
supplying electrical stimulation to a selected site of a patient adjacent, around or in one of the sacral nerve or branches or portions thereof, the pudendal nerve or branches or portions thereof, the hypogastric nerve or branches or portions thereof, the prostatic plexus nerve or branches or portions thereof, the sacral splanchnic nerve or branches or portions thereof, or the pelvic splanchnic nerve or branches or portions thereof in an amount or degree sufficient to provide at least partial relief from pain suffered as a result of one or more of prostatitis, prostatalgia and prostatodynia.

37. The method of claim 36, further comprising implanting the tip of at least one medical electrical lead at the site and transmitting electrical impulses to the at least one lead such that the at least one lead carries electrical impulses to stimulate one or more nerves, nerve branches or nerve portions to induce relief from pain.
